(19) United States
(12) Patent Application Publication
(10) Pub. No.: US 2010/0298833 A1
(43) Pub. Date: Nov. 25, 2010

(54) SYSTEMS, METHODS AND DEVICES FOR PREPARING A KNEE JOINT FOR IMPLANTS IN A KNEE SURGERY

(75) Inventor: Richard Smith, Memphis, TN (US)

Correspondence Address: DIANA HOUSTON;SMITH & NEPHEW, INC., 1450 BROOKS ROAD, MEMPHIS, TN, 38116, US

(73) Assignee: Smith & Nephew, Inc., Memphis, TN (US)

(21) Appl. No.: 12/663,493

(22) PCT Filed: Jun. 9, 2008
(86) PCT No.: PCT/US08/66321

§ 371 Date: Jun. 24, 2010

Related U.S. Application Data
(60) Provisional application No. 60/942,604, filed on Jun. 7, 2007.

(51) Int. Cl. A61B 17/16

(52) U.S. Cl. 606/80

(57) ABSTRACT

A reamer for reaming a joint between at least two bones comprises a housing, a plurality of cutting blades, and a power source. The housing is configured to be inserted within the joint. The plurality of cutting blades is configured to couple to the housing. The plurality of cutting blades creates a cutting surface. The power source is coupled to the housing and configured to deliver motion to the cutting blades such that the cutting blades cut at least one bone of the at least two bones. The method of resurfacing a bone comprises the steps of placing a reamer substantially within a bone joint. The reamer has a housing and a plurality of cutting blades. The cutting blades are configured to bear upon a bone surface. Motive force is delivered to the housing. The motive force drives the plurality of cutting blades to ream the bone surface.

### CROSS-REFERENCE TO RELATED APPLICATIONS

This application claims the benefit of U.S. Provisional Application No. 60/942,604, filed Jun. 7, 2007 the disclosure of which is hereby incorporated by reference.

### BACKGROUND OF THE INVENTION

1. Field of the Invention

This invention relates generally to implants and processes in joint surgery, particularly knee surgery, and more particularly tibial preparation. In certain embodiments, the invention relates to tibial resurfacing guided by soft tissue.

2. Related Art

Tissue guided knee replacement requires instrumentation that is quite different from conventional knee replacement instrumentation due to minimal exposure, minimal bone and cartilage to be removed and soft tissue based resection. In the past, knee implants were fitted to a patient by the surgeon making measured resections by using fixed cutting blocks or by using a medial and lateral jig to control the reaming surface or mill type cutter.

Tissue guided total knee arthroplasty (TKA) requires a reaming tool to remove condylar cartilage and bone while not disturbing the natural kinematics of the patients knee. The reaming device should be small enough to fit in knee joint via an MIS approach and located on the prepared proximal tibia below the unprepared distal femur. The reaming device should be able to power itself or be powered by an external source. Finally the device should be able to function while being precisely manipulated by the surgeon performing the procedure.

With respect to the power systems for these tools, surgical motorized power systems of the past were a large control box wired into a motor via a long cable. The motor speed controls were either integral to the control box, in the motor hand piece or foot pedal operated. These designs generally require the surgeon to hold the motor while performing the procedure.

Past and current tissue guided surgery focuses on separate implants to address bi & tri-compartment knee osteoarthritis. Instrumentation is focused on reaming technology only and does not focus much on resection based instrumentation.

### SUMMARY

It is in view of the above problems that the present invention was developed. An embodiment may include a reamer for reaming a joint between at least two bones comprises a housing, a plurality of cutting blades, and a power source. The housing is configured to be inserted within the joint. The plurality of cutting blades is configured to couple to the housing. The plurality of cutting blades creates a cutting surface. The power source is coupled to the housing and configured to deliver motion to the cutting blades such that the cutting blades cut at least one bone of the at least two bones.

In one aspect of the invention, the cutting surface is a planar surface.

In another aspect of the invention, the power source is coupled to the housing through a flexible drive shaft.

In yet another aspect of the invention, the cutting blades are barrel cutters.

Another aspect of the invention includes cutting blades oscillating in a first direction.

In another aspect of the invention, teeth on the cutting blades are oriented obliquely to the first direction.

In yet another aspect of the invention, the teeth have a V shaped cross section in order to cut in a forward and backward direction.

Another aspect of the invention includes a lavage port configured to lavage biomatter from the joint.

In another aspect of the invention, a crankshaft configured to oscillate the cutting blades.

In yet another aspect of the invention, the reamer reams at least two bones simultaneously.

Another aspect includes the power source coupled to a patient.

Another aspect of the invention provides a method of resurfacing a bone comprises the steps of placing a reamer substantially within a bone joint. The reamer has a housing and a plurality of cutting blades. The cutting blades are configured to bear upon a bone surface. Motive force is delivered to the housing. The motive force drives the plurality of cutting blades to ream the bone surface.

In another aspect of the invention, the cutting blades form a planar cutting surface.

In yet another aspect of the invention, the power is delivered to the housing through a flexible drive shaft.

In one aspect, the cutting blades are rotated.

Alternatively, the cutting blades are oscillated in a first direction.

In yet another aspect, teeth on the cutting blades are oriented obliquely to the first direction.

In yet another aspect of the invention, the teeth have a V shaped cross section in order to cut in a forward and backward direction.

Another aspect of the invention provides the step of lavaging the joint while the reamer is reaming

Yet another aspect provides the step of reaming at least two bones simultaneously.

Additionally, an aspect may provide the step of mounting a power source on a patient.

Further features, aspects, and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated in and form a part of the specification, illustrate embodiments of the present invention and together with the description, serve to explain the principles of the invention. In the drawings:

FIG. 1 is an example of a straight edge barrel reamer according to an aspect of the invention;

FIG. 2 is an example of a modular bone reamer according to an aspect of the invention;

FIG. 3 is an example of an alternating bone reamer including a plurality of reamer blades;

FIG. 4 is an example of an embodiment of one of the reamer blades of FIG. 3;

FIG. 5 is an example of an embodiment of two alternating reamer blades of FIG. 3 depicting a reverse tooth pattern;

FIG. 6 is an example of an embodiment of a crankshaft for driving the alternating reamer blades of FIG. 5;

FIG. 7 is an example of a partial view of the reamer blades of FIG. 5 mounted on the crankshaft of FIG. 6;

FIG. 8 is an example of a partially assembled modular reamer according to an aspect of the invention;

FIG. 9 is an example of an embodiment of a cartridge of a modular reamer;

FIG. 10 is an example of parts of an embodiment of a modular housing;

FIG. 11 is an example of an embodiment of a pair of reamers attached to a motor mounted on a leg; and

FIGS. 12 through 18 are embodiments of reamers and power sources.

### DETAILED DESCRIPTION OF THE EMBODIMENTS

Currently most paradigms of tissue guided knee surgery impose the use of separate implants to address bi or tri-compartment disease. This is usually seen as a patello-femoral joint (PFJ) implant and two unicompartmental implants or a combination of the implants. By using a monolithic implant combined with femoral preparation by tissue guided techniques as well as traditional measured resection advantages of both may be realized

A monolithic femoral component may be used to address bi or tri-compartment disease that is instrumented to the femur using both tissue guided reaming as well as measured resection. Less bone and cartilage may be removed using tissue guided reaming. The system may better restore the patient's natural kinematics. The monolithic femoral component can address bi compartment by replacing either the PFJ and either the medial or lateral condyle with a material thickness of 3 mm to 6 mm in the distal and posterior condylar regions which transitions to normal femoral implant thickness. The same design would be used for tri-compartmental disease except the implant would cover both condyles and the PFJ.

The implant would be instrumented beginning with tissue guided reaming on one or both femoral condyles (after bi-uni tibial plateau resection). Once the required amount of tissue has been reamed from the posterior and distal condylar regions, reaching slightly onto the anterior cortex, asymmetric unicompartmental implant trials that match the amount of tissue removed (3 mm to 6 mm thick) are placed onto the femur. The monolithic femoral implant trial may be sized and placed on the femur. Joint line and balance are reassessed. If all is correct then the surgeon must assess if the patella should be resurfaced. If so then the patella is resurfaced and joint line and balance is reassessed. If the balance and the joint line are proper, the monolithic femoral implant is implanted (possibly with a femoral unicompartmental implant) and with two tibial unicompartmental-implants.

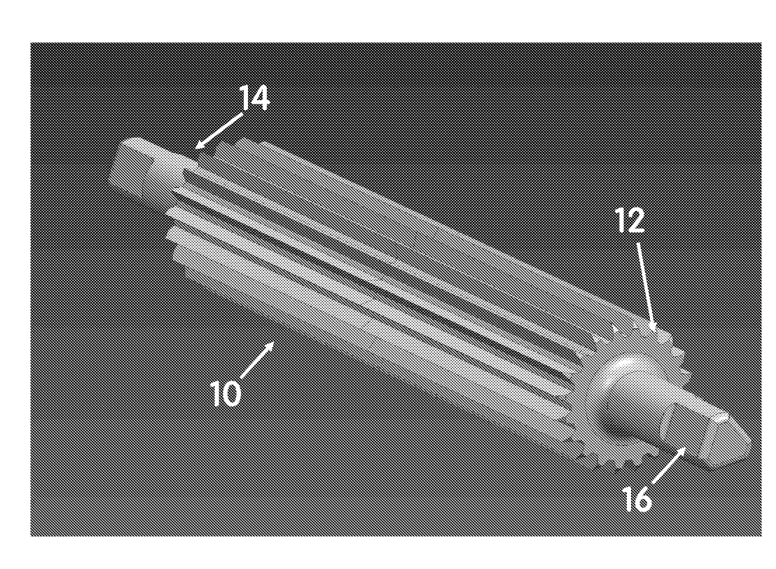

Referring to the accompanying drawings in which like reference numbers indicate like elements, FIG. 1 illustrates an example of a straight edge barrel reamer 10 according to an aspect of the invention. The barrel reamer 10 may be used in a modular reamer. The modular reamer may ream condylar cartilage and bone using the barrel reamer 10. Because the device is modular, in-vivo assembly, multiple choices of reaming cartridges and disposable reamer cartridges may be used.

The straight edge reamer 10 includes cutting teeth 12 located along the circumference of the reamer 10 and extending along the axis of the reamer 10. A shaft 14 extends the length of the reamer 10. The teeth 12 extend radially outward from the shaft 14 of the reamer 10. At the axial ends of the shaft 14, positive mating surfaces 16 may connect the reamer 10 to a drive mechanism. The positive mating surfaces 16, in one embodiment, may be flats along the circumference of the shaft 14.

While the teeth 12 of the current embodiment extend axially along the shaft 14, other embodiments may include teeth that extend both axially and circumferentially along the shaft such that the teeth spiral along the length of the shaft. Similarly, while the teeth have a cross section that is generally triangular in this embodiment, the cross section of the teeth may have other shapes. Such changes may allow the teeth to better move debris along the shaft, cut in only one direction, cut in both directions, or balance forces along the length of the shaft.

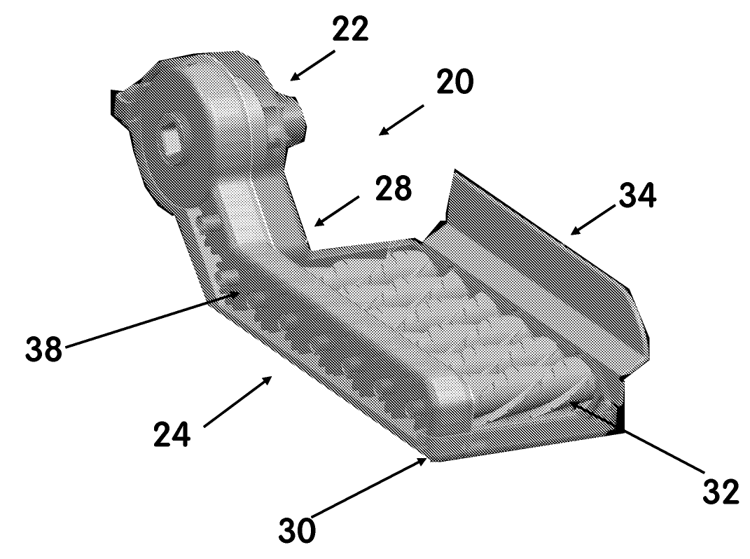

Turning now to FIG. 2, FIG. 2 is an example of a modular bone reamer 20 according to an aspect of the invention. The reamer 20 includes a drive component 22 connected to a reaming component 24 through a connection 28. The drive component 22 is a multiple use component that contains the gears and connections necessary to attach power to the reaming component 24 and distribute the power to the reaming component 24. The reaming component 24 includes a cartridge 30 supporting a plurality of barrel cutters 32 and a tissue protector 34. Gears 38 connect the drive component 22 to the barrel cutters 32. Further components may include but are not limited to fixation components and distraction components.

The drive component 22 is attached to the modular reaming component 24 via a positive locking mating surface that allows the drive component's gears 38 to mesh with those of the drive component 22 through the connection 28. Once the two components have been secured together a drive shaft may be attached to the drive component 22 and locked into place. The drive shaft is attached to a drive motor that powers the assembly. Once the components have been assembled the modular reamer 20 is placed in the joint where the reaming barrel cutters 32 may contact the condylar cartilage. Once the reamer 20 has been placed in the correct location the motor is energized and the drive shaft begins to turn drive gears in the drive component 22. This, in turn, turns the gears 38 in the modular reamer cartridge 24 which turns the barrel cutters 32. The turning of the barrel cutters 32 removes the cartilage and bone of the condyle.

The modular bone reamer 20 is designed to fit into a knee joint and ream either the proximal tibia or the distal femur and can be located in either the medial or lateral compartments or both compartments simultaneously.

The body of the drive component 22 can be either straight or angled to allow for single condylar reaming or bi-condylar reaming or custom fit for patient anatomical needs. The reaming surface is comprised of rotating cylindrical cutters that may have multiple methods of operation including opposite directions of rotation or same directions of rotation if idlers are employed in the drive assembly. The actual reaming blades are straight barrels with straight cutting edges that are parallel to the axis of the barrel or they could be conventional helical fluted cutters. Vast cutter geometries may be used with certain designs most suitable for particular applications. Rotation is achieved by powering directly meshed spur gears that are attached to the cutters. This method of driving the cutters helps balance the reaming forces in the joint and maintain as much efficiency as possible by not having idler gears.

By making the reaming device modular it could be possible to reduce costs to the patient by having a multi-use drive component where the patient would not have to incur the cost of a single use device. This allows for a less expensive reaming component to be a single use device that attaches to the drive component for each surgical procedure. This configuration also increases the number of reaming solutions by having a reusable drive body that multiple designs of reaming cartridges may attach and allowing the reaming system to meet several different needs of the doctors and patients. The possible different types of devices that could be attached to the drive component are reaming carriages with different number of cutters, different lengths of cutters, alternating cutters, belt cutters and different configurations of barrel cutters. Finally the modularity of the reamer will allow the surgeon to assemble the device in-vivo and/or in-situ thus allowing for a smaller incision on the patient.

Other modular reamers could use different cutting geometry such as alternating cutting blades or a belt like cutting surface. Other mechanical means, in addition to the drive shaft, could be used to transfer power/energy to the reamers. The cartridge may also be modular so that the barrel cutters may be added or located in various stages (i.e. click on barrel sections that allow the reamer to adjust from a single barrel reamer to a plurality of reamers, oriented in different directions).

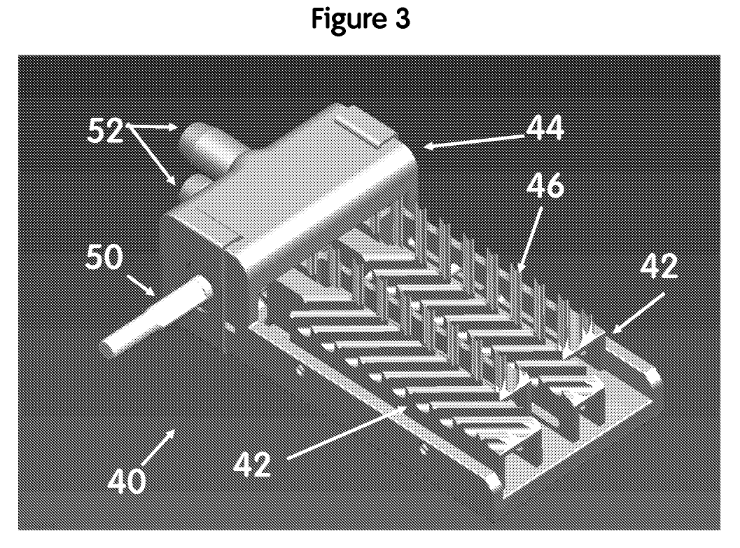

Turning now to FIG. 3, FIG. 3 is an example of an alternating bone reamer 40 including a plurality of reamer blades 42. The balanced alternating bone reamer 40 is designed to ream femoral condylar cartilage and bone by the use of an even number of alternating reverse toothed cutting blades that reciprocate opposite to each other during the reaming process. This function of the even numbered alternating cutting reamers 42 resects the tissue while not upsetting the natural kinematics of the patient's knee.

The balanced alternating bone reamer 40 is designed in this embodiment to fit into the knee joint and ream either the proximal tibia or the distal femur and can be located in either the medial or lateral compartments or both compartments simultaneously. The balanced alternating bone reamer 40 is comprised of a reamer housing 44 and even number of alternating reaming blades 42 with a reverse tooth pattern 48, a crank shaft 50 that moves the reaming blades in an alternating manner and a lavage portals 52 located on the housing 44 and allowing the flow of fluid under and through the blades 42. Further components may include but are not limited to tissue protectors, fixation features and distraction features.

The net external force of the cutting blade system is essentially zero, and thus a balanced system. As half of the blades 42 are cutting in a forward direction, the other half of the blades 42 are cutting in a backward direction. Thus, the force of the forward moving blades 42 cutting against the bone and cartilage are approximately equal to the forces exerted from the bone and cartilage acting upon the blades 42 moving in the opposite direction. Such a setup may keep the bone reamer 40 from chattering or moving within the joint.

The housing 44 is modular to allow for the internal parts (crankshaft, gearing, reaming blades 42 and bushings) to be assembled and inserted. The major portions of the housing 44 are positively locked together once assembled. The anterior portion of the modular housing can have from two to five power ports that allow power to be applied to the crankshaft from the medial, lateral, anterior, superior and inferior aspects of the reamer 40 or any combination of the five aspects. Alternatively, the reamer may have internal mating features that would allow the optional connection of power from any of an assortment of directions, (eg medial or lateral or anterior). This would then embody a single reamer with application to different compartments or surgeon preference on how to connect power.

Other embodiments may include an odd number of reaming blades. The blades may have straight teeth as opposed to oblique. The blade tooth geometry may be varied or different. The blades may even become an adequately roughened surface. The blades may articulate with a cam shaft and spring/bumper resistance to create the opposing forces to the cam shaft. The blades may articulate with an internal cam shaft in place of the crankshaft that would articulate on a closed journal in the blade as opposed to the open journal this design has. The crankshaft may be modular. Instead of alternating reaming blades, the reamer may alternate superiorly facing saw blades.

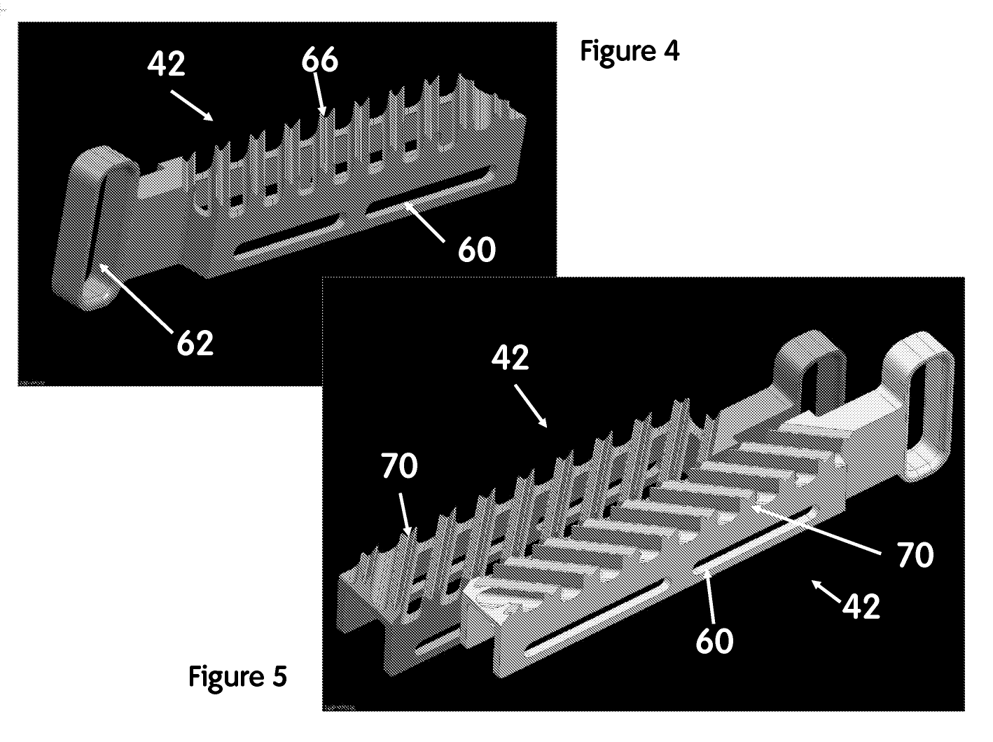

Turning now to FIG. 4, FIG. 4 is an example of an embodiment of one of the reamer blades of FIG. 3. The blades 42 are kept on in place inferiorly and superiorly by two or more transverse cross bars that intersect the reaming blades 42 through transverse slots 60 located on the side of the reaming blade 42. The blades 42 are constrained transversely by the sides of the housing. A crankshaft slot 62 receives the crankshaft, as described below.

Teeth of the reamer blade 42 have a generally V shaped cross section. The V-shaped cross section of the teeth 66 allow the teeth to cut in both a forward and backward direction. By separating the teeth 66 from each other, bone material may slide between the teeth 66 and be flushed out by the lavage. The teeth 66, in this embodiment, are also oriented obliquely relative to the direction of the movement of the blade 42. Both the cross section and orientation of the blade 42 may be adjusted in other embodiments.

Turning now to FIG. 5, FIG. 5 is an example of an embodiment of two alternating reamer blades 42 of FIG. 3 depicting a reverse tooth pattern. The reamer blades 42 have reverse oblique oriented teeth 70. One reamer blade 42 is forward positioned and the other is reverse positioned showing how the blades 42 reciprocate. The teeth 70 are obliquely oriented in opposite directions which may help to minimize the external forces by balancing out the lateral forces in opposite directions between the blades 42. In other words the tooth pattern of one blade is opposite to that of the blade located next to it. The cutting teeth are oblique to the long centerline of the blades with a tissue evacuation portion located between each tooth.

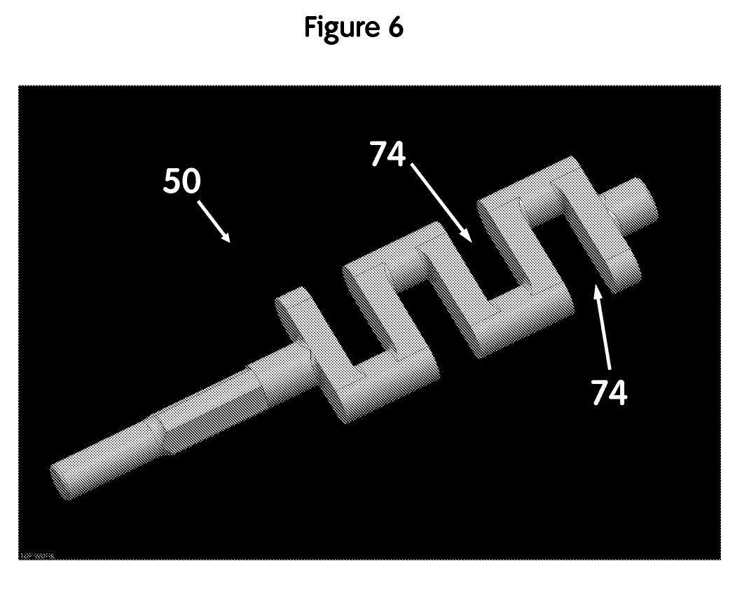

Turning now to FIG. 6, FIG. 6 is an example of an embodiment of a crankshaft for driving the alternating reamer blades of FIG. 5. The crankshaft includes a plurality of journals 74 to receive blades. The crank shaft 50 that drives the reaming blades 42 is powered by a power system that delivers power to the reamer 40 either medially, laterally or anteriorly. The design of the crankshaft 50 itself is a one piece “snake” like design that allows the reaming blades to be assembled to the crankshaft 50 by sliding them over either one of the ends of the crankshaft 50 and placing them in their respective journals 74. The journals 74 of the crankshaft 50 and internal grooves located internally to the anterior portion of the housing. Power can either be applied directly to the crankshaft or can be applied to drive gears that mesh with the crankshaft.

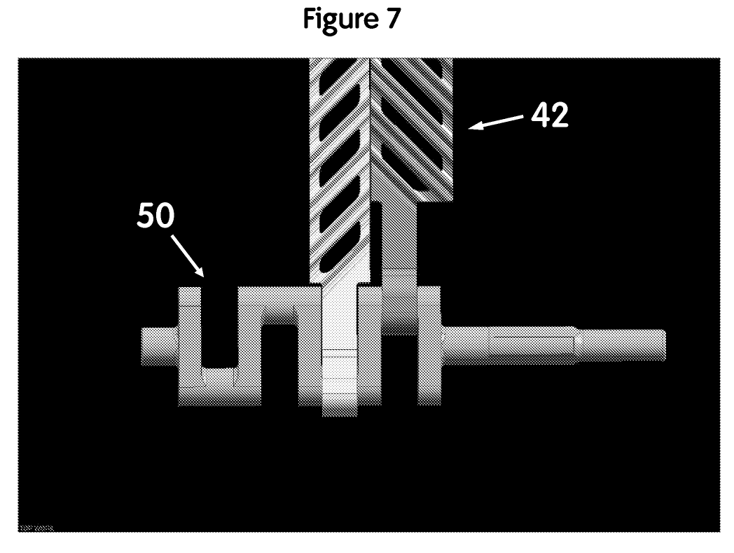

Turning now to FIG. 7, FIG. 7 is an example of a partial view of the reamer blades of FIG. 5 mounted on the crankshaft of FIG. 6. As the crankshaft 50 is rotated, blades 42 reciprocate forward and backward. As the crankshaft 50 rotates, the journals rotate within the crankshaft slot to allow for the reciprocating motion to only move in one direction. Thus the length of the crankshaft slot is approximately equal to the diameter of the journals as the crankshaft is rotated.

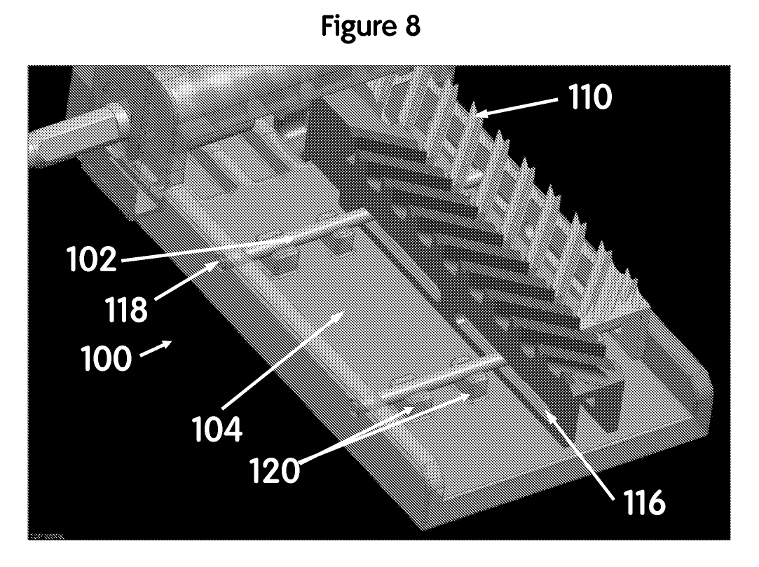

Turning now to FIG. 8, FIG. 8 is an example of a partially assembled modular reamer 100 according to an aspect of the invention. Cross bars 102 in a housing 104 support blades 110. The bars 102 extend laterally through slots 116 in the blades 110. The cross bars 102 are supported by transverse holes 118 located in the side of the reamer housing 104 and several supports 120 located inferior and internal to the reaming blades 110. The blades 110 are raised above the sides of the housing so that the housing sides do not act as a depth stop. If a depth stop is desired in reaming, the height of the blades can be a specific height above the side of the housings. The distance from the top of the blades to the top of the sides of the housing will determine the depth of reaming allowed by the reamer.

While this embodiment includes a housing that has a bottom portion, other embodiments may not have a bottom portion and the blades may be supported from the housing sides. The blades may include cutting teeth on both the top and bottom of the reamer. In such an embodiment, the blades may cut both above and below the reamer. Thus, when put in a joint like the knee, the reamer may be configured to cut cartilage and bone on both sides of the reamer, thereby cutting both the femur and the tibia at the same time. Such an embodiment may better align the cutting surfaces between the two bones and may also be used to effectively gauge the depth of the resurfacing of the bone on both sides of the implant.

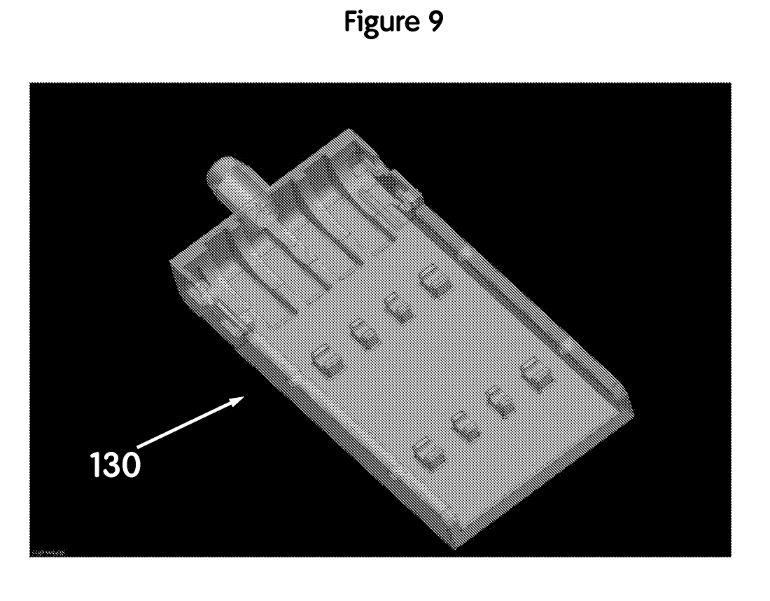

Turning now to FIG. 9, FIG. 9 is an example of an embodiment of a cartridge of a modular reamer 130. While the embodiment shows four supports for each crossbar for four cutting blades, fewer or additional supports may be added. Additionally, the embodiments are not limited to only four blades.

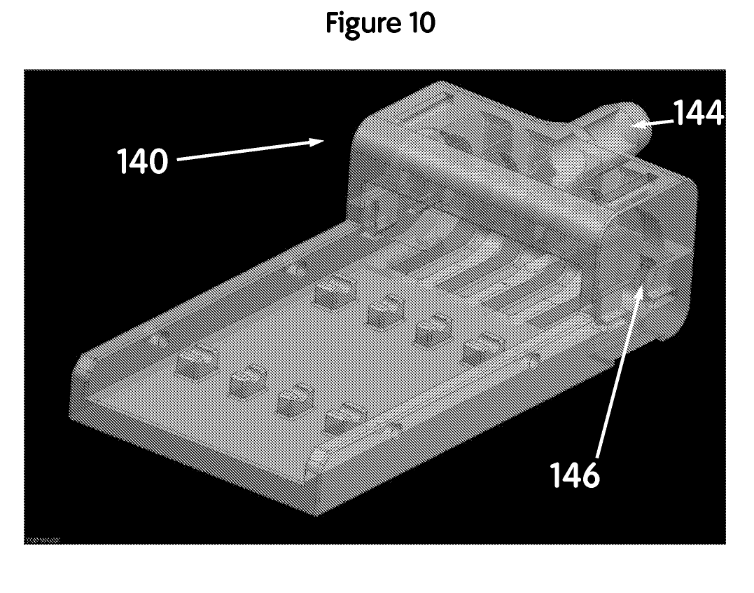

Turning now to FIG. 10, FIG. 10 is an example of parts of an embodiment of a modular housing 140. The housing includes a lavage port 144 and a crankshaft guide 146. The housing supports the lavage ports 144 and the crankshaft for the blades. Bone and cartilage, when cut free, may flow under the blades through the lavage ports 144 and out of the joint.

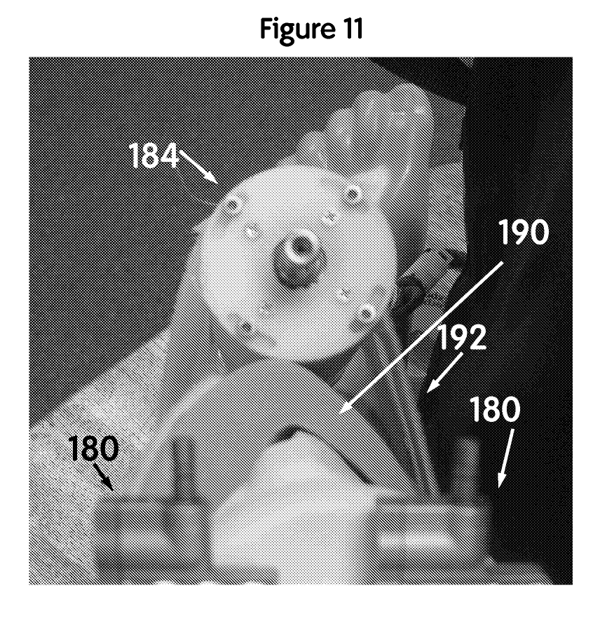

Turning now to FIG. 11, FIG. 11 is an example of an embodiment of a pair of reamers 180 attached to a motor 184 mounted on a leg 190. The motor may be attached with Velcro 190 or other fixation means. Drive shafts may be connected from the motor 184 to the reamers 180.

When performing various surgical procedures where the instrument(s) require energy from an external source (motor), many times surgeons require the use of both hands to manipulate the patient or the instrument. By including the power source (battery(s) or transformer), motor controls (I.e trigger for speed and/or motor control) and motor in a hands free device with or without gearing onto a patients leg, the surgeon is free to use both hands to manipulate the patients anatomy or the instrumentation in the desired manor. While power sources (and possibly controls) have been isolated from the instrument and delivered to the motor and the instrument previously by cables, the motor has generally remained attached to the instrument. The motor may be heavy (limiting agility and responsiveness of the instrument) and may limit access to the surgical site for instruments based upon size of the motor and instrument. Thus, being able to isolate the motor, control and power source from the instrument may increase access and increase surgeon agility as well as increase instrument tactile instrument proprioception. An appendage attached surgical instrument motor, gearing, power supply, motor control and housing may address some of these issues.

In one embodiment, the complete appendage assembly is comprised of a battery(s), one or two DC motors, motor control circuitry, a speed control device (potentiometer or like) a housing contoured to fit the anatomy of a human leg or other appendage, a gel pad and Velcro securing straps. The assembled device would be strapped to the patient's leg and attached to the surgical instrument via flexible or rigid drive shafts. Once the motor is energized it would power the surgical instrument at various speeds and torques depending on the input of the surgeon via the speed control device that in integral to the leg assembly.

The device would be mounted mid-shaft of the proximal or distal portion of the entire appendage directly below or above the joint that the instrument in involved in. It would be connected the appendage by Velcro straps or similar types of devices as well as padding between the housing and the patient to protect the patient from impact and heat from the motor as well as to dampen an vibration caused by the motor or instrument. The device would be connected to the instrument requiring power by either a flexible or rigid drive shaft. Devices that could take advantage of this type of device would include but not be limited to reamers, drills, burrs, saws and power distraction or reduction devices.

The assembly could be attached to the thigh or any appendage of the patient. The device could be strapped to the surgeon. The device could pull power from an AC outlet or pneumatic system instead of the battery. The device could be divided into modules and only the motor and possibly the speed control is attached to the patient's leg with the other modules being compact and located on the table with the patient or on a side table. This would require the motor and control to be attached via a cable. A final possibility is that motor and control circuitry is attached to the leg and either powered by a battery or power cord. The difference being the speed control is located in the surgeon's hand and sends the control signals via Blue Tooth or similar technology. The instrument could be handheld and the controls and power source could be located on the patient's or doctor's appendage or body.

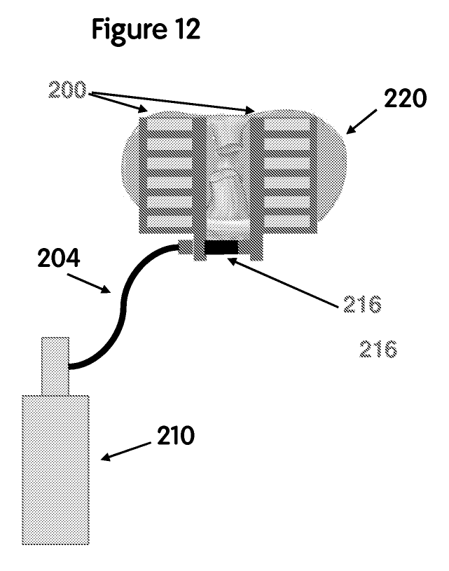

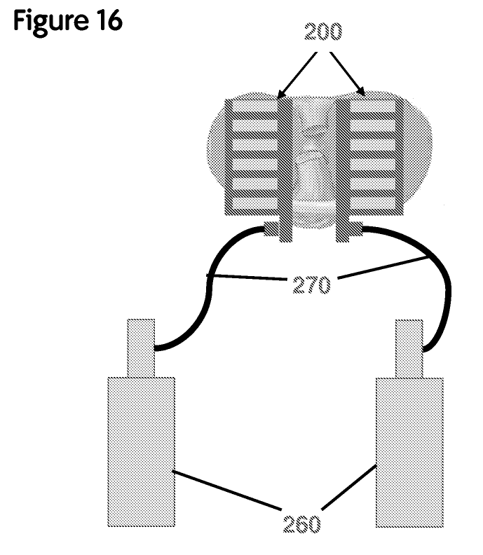

Turning now to FIGS. 12 through 18, FIGS. 12 through 18 are embodiments of reamers and power sources. FIG. 12 is an example of an embodiment of a pair of reamers 200 driven through a single drive shaft 204 and motor 210. A coupling 216 couples the reamers 200 to each other. The reamers 200 are placed within the joint over a tibia 220. The coupling 216 allows for energy to transfer between the reamers 200 while maintaining the middle portions of the tibia where the cruciate ligaments cross. A single power supply 210 and single flexible shaft allow for minimal amounts of power devices within the joint. This may be beneficial for MIS approaches where minimal incisions are made. However, chatter from one reamer may adversely effect the other reamer.

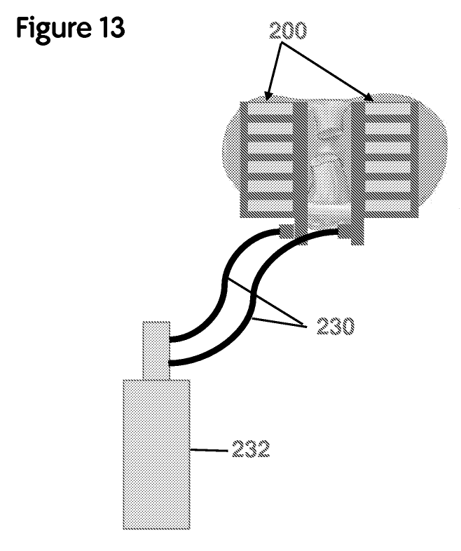

Turning now to FIG. 13, FIG. 13 is an example of an embodiment of a pair of reamers 200 being driven through a pair of flexible drive shafts 230 from a motor 232. The embodiment requires an additional flexible drive shaft through the incision, but may help to eliminate chatter between the reamers. Additionally, the set of shafts 230 may allow for independent shimming of the different compartments.

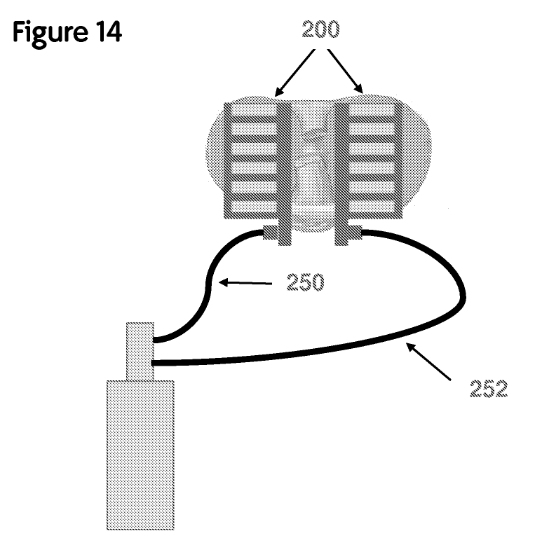

Turning now to FIG. 14, FIG. 14 is an example of an embodiment of a pair of reamers 200 being driven by a single motor through a pair of drive shafts 250 and 252 each extending through different incisions. The drive shaft 250 may extend through a lateral incision while the drive shaft 252 may extend through a lateral incision. The embodiment may allow for better clearance through the primary medial incision while making a small lateral incision. However, an additional incision is required.

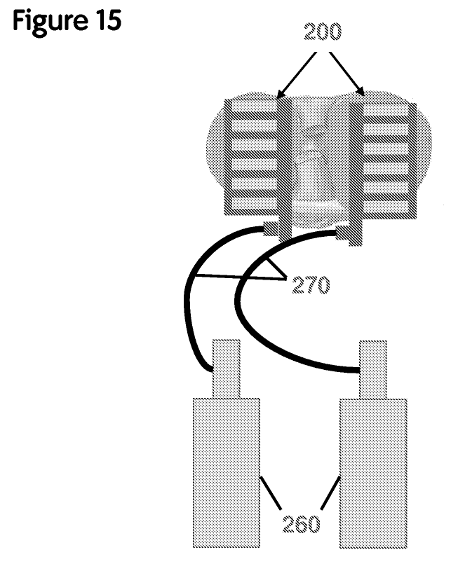

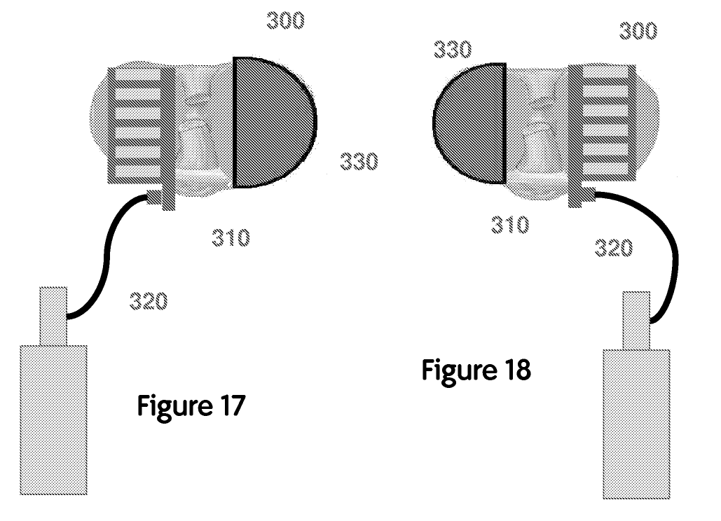

Turning now to FIG. 15, FIG. 15 is an example of an embodiment of a pair of reamers 200 being driven by a pair of motors 260 through a pair of drive shafts 270. FIG. 17 is an example of an embodiment of a pair of reamers 200 being driven by a pair of motors 260 through a pair of drive shafts 260 each extending through different incisions, similar to the embodiment of FIG. 14.

Turning now to FIGS. 18 and 19, the figures are an example of an embodiment of a single reamer 300 being used serially to prepare the lateral and medial sides of a tibia 310. A single drive shaft 320 may extend through the medial incision or through a lateral incision in order to prepare the lateral side. Inserts 330 may be used to support the other compartment when one compartment is being reamed. Control for each condyle may be better, but additional time would be needed to prepare both condyles.

In view of the foregoing, it will be seen that several advantages of the invention may be achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A reamer for reaming a joint between at least two bones, comprising:
a. a housing configured to be inserted within the joint;
b. a plurality of cutting blades configured to couple to the housing, the plurality of cutting blades creating a cutting surface;
c. a power source coupled to the housing and configured to deliver motion to the cutting blades such that the cutting blades cut at least one bone of the at least two bones
wherein the plurality of cutting blades are configured to be reciprocated along an axis, a first of the plurality of cutting blades moving in a first forward direction along the axis and a second of the plurality of cutting blades moving in a second backward direction along the axis such that the first and second direction are oppositely moving at the same time.

2. The reamer of claim 1, wherein the cutting surface is a planar surface.

3. The reamer of claim 1, wherein the power source is coupled to the housing through a flexible drive shaft.

4. The reamer of claim 1, wherein the cutting blades comprise cutting surfaces extending across a planar cutting surface of the cutting blades.

5. The reamer of claim 4, wherein the cutting surfaces comprise teeth aligned along the planar cutting surface and the cutting blade is configured to reciprocate the teeth along the axis.

6. The reamer of claim 5, wherein teeth on the cutting blades are oriented obliquely to the axis.

7. The reamer of claim 5, wherein the teeth have a V shaped cross section in order to cut in a forward and backward direction.

8. The reamer of claim 1, further comprising a lavage port configured to lavage biomatter from the joint.

9. The reamer of claim 5, further comprising a crankshaft configured to reciprocate the cutting blades.

10. The reamer of claim 1, wherein the reamer reams at least two bones simultaneously.

11. The reamer of claim 1, wherein the power source is coupled to a patient.

12. A method of resurfacing a bone, comprising the steps of:
a. placing a reamer substantially within a bone joint, the reamer having a housing and a plurality of cutting blades, the cutting blades configured to bear upon a bone surface; and
b. delivering motive force to the housing, the motive force driving the plurality of cutting blades to ream the bone surface wherein the motive force reciprocates the plurality of cutting blades, a first of the plurality of cutting blades moving in a first forward direction along the axis and a second of the plurality of cutting blades moving in a second backward direction along the axis such that the first and second direction are oppositely moving at the same time.

13. The method of claim 12, wherein the cutting blades form a planar cutting surface.

14. The method of claim 12, wherein the power is delivered to the housing through a flexible drive shaft.

15. The method of claim 12, wherein the cutting blades have cutting surfaces extending across a planar cutting surface.

16. The method of claim 12, wherein the cutting blades comprise teeth aligned along the planar cutting surface and the cutting blade is configured to reciprocate the teeth along the axis.

17. The method of claim 16, wherein teeth on the cutting blades are oriented obliquely to the axis.

18. The method of claim 16, wherein the teeth have a V shaped cross section in order to cut in a forward and backward direction.

19. The method of claim 12, further comprising the step of lavaging the joint while the reamer is reaming.

20. The method of claim 12, further comprising the step of reaming at least two bones simultaneously.

21. The method of claim 12, comprising the step of mounting a power source on a patient.
